(19) United States
(12) Patent Application Publication
(10) Pub. No.: US 2026/0014094 A1
(43) Pub. Date: Jan. 15, 2026

(54) METHOD FOR PRODUCING A LIQUID COMPOSITION INCLUDING A NANOPARTICLE, AND FORMULATION THEREOF

(71) Applicants: GLOBAL LIFE SCIENCES SOLUTIONS CANADA ULC, Vancouver (CA); GLOBAL LIFE SCIENCES SOLUTIONS UK LIMITED, Buckinghamshire (GB)

(72) Inventors: Pierrot HARVIE, Vancouver (CA); Roman Petro KONDRA, Vancouver (CA)

(21) Appl. No.: 19/337,639

(22) Filed: Sep. 23, 2025

Related U.S. Application Data
(63) Continuation of application No. PCT/EP2025/061273, filed on Apr. 24, 2025.
(60) Provisional application No. 63/638,647, filed on Apr. 25, 2024.

(51) Int. Cl. A61K 9/51; A61K 31/7105; A61K 31/711; A61K 31/713; A61K 47/08

(52) U.S. Cl. CPC A61K 9/5192; A61K 9/5123; A61K 31/7105; A61K 31/711; A61K 31/713; A61K 47/08

(57) ABSTRACT

A method is provided for producing a composition that includes a nanoparticle with at least one nucleic acid and at least one ionizable lipid. The method includes introducing a first composition that includes the at least one nucleic acid and a second composition that includes the at least one ionizable lipid into at least one reactor. The first composition and the second composition are introduced at a first flow rate and a second flow rate, respectively, therefore generating the nanoparticle in a reaction mixture. The method further includes filtering the nanoparticle from the reaction mixture via a single-pass tangential flow filter at a feed flux to provide a retentate, to produce the composition. The method may also include filtering the retentate through a sterile filtration membrane.

### CROSS REFERENCE TO RELATED APPLICATION

This patent application is a continuation of and claims priority to International patent application PCT/EP2025/061273 filed on Apr. 24, 2025, which further claims the benefit of U.S. Provisional Patent Application No. 63/638,647, filed on Apr. 25, 2024, which is incorporated by reference in its entirety herein.

### FIELD

Embodiments of the present disclosure relate generally to the field of nanoparticles, and, more particularly, to a method for producing a composition comprising nanoparticles. The present disclosure further relates generally to the produced formulations and the related therapeutic and/or diagnostic uses, such as methods involving nanoparticles to deliver one or more therapeutics.

### BACKGROUND

Microparticles and nanoparticles have important applications in biomedicine, pharmacy, medicine, chemical industries, and the like. Many techniques are available for their manufacture.

The effective targeted delivery of biologically active substances such as small molecule drugs, proteins, and nucleic acids represents a continuing challenge. In particular, the delivery of nucleic acids to cells is made difficult by the relative instability and low cell permeability of such species.

Lipid nanoparticles (LNP or LNPs) have proven effective as transport vehicles into cells and/or intracellular compartments for biologically active substances such as small molecule drugs, proteins, and nucleic acids. When LNP are manufactured using traditional manufacturing methods, there may be issues with maintaining potency and reducing toxicity. A variety of LNP have been demonstrated, and while promising for therapeutic applications, improvements in safety, efficacy and specificity are desired.

Typically, tangential flow filtration (TFF) is used in the downstream processing steps for the LNP preparation, the goal of the TFF is to exchange liquid compositions from those required in LNP formation to a biologically compatible, final composition for administration. When filtering LNP using conventional tangential flow filtration (TFF), the LNP must be recirculated multiple times through the filtration system to reach desired concentration and buffer composition. This recirculation requires substantial processing time subjects the LNP to hydrodynamic shear forces, both of which may adversely impact LNP potency.

### BRIEF SUMMARY

Certain embodiments commensurate in scope with the originally claimed subject matter are summarized below. These embodiments are not intended to limit the scope of the claimed subject matter, but rather these embodiments are intended only to provide a brief summary of the possible embodiments. Indeed, the disclosure may encompass a variety of forms that may be similar to or different from the embodiments set forth below.

According to an aspect of the invention, a method is provided for producing a composition that includes a nanoparticle (LNP) with at least one nucleic acid and at least one ionizable lipid, the method including: introducing a first composition that includes the at least one nucleic acid and a second composition that includes the at least one ionizable lipid into at least one reactor, wherein the first composition and the second composition are introduced at a first flow rate and a second flow rate, respectively, therefore generating the nanoparticle in a reaction mixture; and filtering the nanoparticle from the reaction mixture via a single-pass tangential flow filtration system at a feed flux to provide a retentate, to produce the composition.

In embodiments, the method further includes filtering the retentate through a sterile filtration apparatus before or after filtering the nanoparticle from the reaction mixture via the single-pass tangential flow filtration system.

In embodiments, filtering the nanoparticle further includes: concentrating the reaction mixture to provide a concentrated sample; subjecting the concentrated sample to an in-line diafiltration to provide a post diafiltration sample; and concentrating the post diafiltration sample to provide the retentate.

In embodiments, concentrating the reaction mixture including flowing the reaction mixture via the single-pass tangential flow filtration system once and without recirculation.

In embodiments, filtering the nanoparticle includes one or more of flowing, passing, running, or moving the nanoparticle from the reaction mixture through the single-pass tangential flow filter.

In embodiments, the nanoparticles in the reaction mixture are filtered in a batch-wise fashion.

In embodiments, the nanoparticles in the reaction mixture are filtered continuously.

In an embodiment, the at least one nucleic acid includes a nucleic acid. In embodiments, the at least one nucleic acid includes a ribonucleic acid (RNA). In embodiments, the at least one nucleic acid includes a deoxyribonucleic acid (DNA). The nucleic acid may be one or more of linear or circular or closed deoxyribonucleic acid (DNA), linear or circular ribonucleic acid (RNA). In embodiments, the nucleic acid includes a circular RNA, messenger RNA (mRNA), transfer RNA (tRNA), ribosomal RNA (rRNA), small nuclear RNA (snRNA), microRNA (miRNA), small interfering RNA (siRNA), self-amplifying RNA (saRNA), guide or targeting RNA, or a combination thereof. In embodiments, the at least one nucleic acid includes a self-amplifying RNA (saRNA) or a messenger RNA (mRNA). In embodiments, the nucleic acid includes proteins or enhancers associated with an application of the nucleic acid.

In embodiments, the second composition further includes one or more of a phospholipid, sterol, or a stabilizing agent.

In embodiments, the second composition comprises about 10-75 Mol % ionizable lipid, about 1-75 Mol % phospholipid, about 4-70 Mol % sterol, and about 0-3 Mol % stabilizer, wherein the total mol % of components in the second composition is 100 mol %.

In embodiments, the second composition comprises about 25-50 Mol % ionizable lipid, about 10-55 Mol % phospholipid, about 20-40 Mol % sterol, and about 0-3 Mol % stabilizer, wherein the total mol % of components in the second composition is 100 mol %.

In embodiments, the first flow rate is different from the second flow rate.

In embodiments, the feed flux is between 10 L/m²/hour and 200 L/m²/hour.

In an embodiment, the predetermined flux includes a flux for the LNP composition. In one example, the predetermined fluxes are between 10 L/m²/hour (LMH) and 200 LMH.

According to another aspect of the invention, a system for producing a composition is provided. The system includes a reactor and a single-pass tangential filtration system. The reactor is configured to combine a first composition at a first flow rate with a second composition at a second flow rate to generate a nanoparticle in a reaction mixture, wherein the first composition comprises at least one nucleic acid and the second composition comprises at least one ionizable lipid. The single-pass tangential filtration system is configured to filter the nanoparticle from the reaction mixture at a feed flux to provide a retentate.

In embodiments, the system further includes a sterile filtration apparatus in fluidic communication with the single-pass tangential flow filtration system and configured to filter the retentate provided from the single-pass tangential flow filtration system.

In embodiments, the single-pass tangential flow filtration system is configured to flow the reaction mixture through once and without recirculation.

In embodiments, the system further includes a first fluid source includes a first liquid composition having at least one nucleic acid. The second fluid source includes a second liquid composition having at least one ionizable lipid.

In embodiments, the single-pass tangential flow filtration system is configured to: concentrate the reaction mixture to provide a concentrated sample; flow the concentrated sample via an in-line diafiltration to provide a post diafiltration sample; and concentrate the post diafiltration sample to provide the retentate.

In embodiments, the single-pass tangential flow filtration system includes a concentration module and a diafiltration module.

In embodiments, the single-pass tangential flow filtration system is configured to filter the nanoparticles from the reaction mixture in a batch-wise fashion.

In embodiments, the single-pass tangential flow filtration system is configured to filter the nanoparticle from the reaction mixture continuously.

In embodiments, the at least one nucleic acid includes a linear or a circular ribonucleic acid (RNA) or deoxyribonucleic Acid (DNA). In embodiments, the at least one nucleic acid includes a circular RNA, messenger RNA (mRNA), transfer RNA (tRNA), ribosomal RNA (rRNA), small nuclear RNA (snRNA), microRNA (miRNA), small interfering RNA (siRNA), a self-amplifying RNA (saRNA), guide or targeting RNA, or combinations thereof. In embodiments, the nucleic acid is a self-amplifying RNA (saRNA) or a messenger RNA (mRNA). In embodiments, the at least one nucleic acid further includes a protein or enhancer associated with the at least one nucleic acid.

In embodiments, the second composition further includes one or more of a phospholipid, sterol, or a stabilizing agent.

In embodiments, the second composition comprises about 10-75 Mol % ionizable lipid, about 1-75 Mol % phospholipid, about 4-70 Mol % sterol, and about 0-3 Mol % stabilizer, wherein the total mol % of components in the second composition is 100 mol %.

In embodiments, the second composition comprises about 25-50 Mol % ionizable lipid, about 10-55 Mol % phospholipid, about 20-40 Mol % sterol, and about 0-3 Mol % stabilizer, wherein the total mol % of components in the second composition is 100 mol %.

In embodiments, the first flow rate is different from the second flow rate.

In embodiments, the feed flux is between 10 L/m²/hour and 200 L/m²/hour.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

The present invention will be better understood from reading the following description of non-limiting embodiments, with reference to the attached drawings, wherein below:

FIG. 1 is a diagram illustrating a general process of producing a liquid composition comprising a nanoparticle (LNP) including at least one nucleic acid and at least one ionizable lipid, according to one example.

FIG. 2 is a graph illustrating the size and polydispersity index of a V02 LNP subjected to the SPTFF method of the instant disclosure, with DSPC as a helper lipid.

FIG. 3 is a graph illustrating encapsulation efficiency and RNA concentration of the corresponding LNP samples in FIG. 2.

FIG. 4 is a graph illustrating comparison of the percentage of spike positive cells per dose transformed of conventional tangential flow filtration and single-pass tangential flow filtration.

FIG. 5 is a graph illustrating the size and polydispersity index of a V02 LNP subjected to the SPTFF method of the instant disclosure, with DOPE as a helper lipid.

FIG. 6 is a graph illustrating encapsulation efficiency and RNA concentration of the corresponding V02 LNP DOPE samples in FIG. 5.

FIG. 7 is a graph illustrating the size and polydispersity index of a V46 LNP subjected to the SPTFF method of the instant disclosure.

FIG. 8 is a graph illustrating encapsulation efficiency and RNA concentration of the corresponding V46 LNP samples in FIG. 7.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “nanoparticle” is defined as a particle comprising more than one component material (for instance lipid or lipids, polymer or polymers, or both lipid and polymer combinations, etc.) that is used to encapsulate a therapeutic material. In some embodiments, the nanoparticle possesses a dimension that is less than 350 nanometers (nm). Nanoparticles include, but are not limited to, lipid nanoparticles (LNP) and polymer nanoparticles. In some embodiments, the term “nanoparticle” and lipid nanoparticles (LNP) may be used interchangeably throughout the current disclosure. In embodiments, a nanoparticle represents the physical organization of the lipid mix composition with the therapeutic agent and among the components. The structural organization of nanoparticles may lead to an aqueous interior with one or more bilayers as in liposomes or it may have a solid interior as in a solid nucleic acid lipid nanoparticle. There may be phospholipid monolayers or bilayers in single or multiple forms. Nanoparticles are between 1 and 1000 nm in diameter.

The term “nucleic acid” refers to any polymeric chain of nucleotides. A nucleic acid may be DNA, RNA, or a combination thereof. In some embodiments, a nucleic acid comprises one or more natural nucleic acid residues. In some embodiments, a nucleic acid comprises of one or more nucleic acid analogs. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55,60,65, 70, 75,80, 85,90,95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long (e.g., 20 to 100, 20 to 500, 20 to 1000, 20 to 2000, or 20 to 5000 or more residues). In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide.

The terms “payload” and “nucleic acid therapeutic” mean the nucleic acid as described above, and any necessary addition components for integration, expression, or interference with the genetic mechanisms of a target cell or cells.

As used herein, “encapsulation” or “encapsulated” may refer to complete, substantial, or partial enclosure, confinement, surrounding, or encasement. As used herein, “encapsulation” may refer to the process of confining an individual nucleic acid molecule within a nanoparticle and/or establishing a physiochemical relationship between an individual nucleic acid molecule and a nanoparticle.

As used herein, “encapsulation efficiency” refers to the amount of a therapeutic and/or prophylactic that becomes part of an LNP, relative to the initial total amount of therapeutic and/or prophylactic used in the preparation of an LNP.

As used herein, the terms “approximately” and “about,” as applied to one or more values of interest, refer to a value that is similar to a stated reference value. In some embodiments, the term “approximately” or “about” refers to a range of values that fall within, for example, 25%, 20%, 10%, 5%, 4%, 3%, 2%, 1%, or less in either direction (greater than or less than) of the stated reference value unless otherwise stated or otherwise evident from the context (except where such number would exceed 100% of a possible value).

In embodiments, lipid nanoparticle (LNP) compositions are configured to encapsulate nucleic acids. The lipid nanoparticle compositions for encapsulating nucleic acids may include an ionizable lipid. The lipid nanoparticle compositions for encapsulating nucleic acids may also include one or more of a structural lipid (also referred to as “helper lipid” or “non-ionizable lipid” in the disclosure), a sterol (also referred to as “neutral lipid” in the disclosure), a stabilizing agent (also referred to as “stabilizer” in the disclosure). The resulting encapsulated LNPs may be used in a variety of applications, particularly medical applications such as oligonucleotide-based therapeutics, e.g., gene therapy, cell therapy.

Any suitable ionizable lipid can be present in the lipid nanoparticle composition and lipid nanoparticle. As used herein, the term “ionizable lipid” refers to a lipid that is cationic or becomes ionizable (protonated) as the pH is lowered below the pKa of the ionizable group of the lipid but is more neutral at higher pH values. At pH values below the pKa, the lipid is then able to associate with negatively charged nucleic acids (e.g., oligonucleotides). Ionizable lipid includes lipids that assume a positive charge on pH decrease from physiological pH, and any of a number of lipid species that carry a net positive charge at a selective pH. In some embodiments, the lipid nanoparticle composition or lipid nanoparticle comprises one or more ionizable lipids, e.g., two or more ionizable lipids, three or more ionizable lipids, or four or more ionizable lipids. In some embodiments, the ionizable lipid includes, but is not limited to, DODMA (1,2-dioleyloxy-3-dimethylaminopropane), DLin-MC3-DMA (O-(Z,Z,Z,Z-heptatriaconta-6,9,26,29-tetraen-19-yl)-4-(N,N-dimethylamino)), DLin-KC2-DMA (2-dilinoleyl-4-dimethylaminocthyl-[1,3]-dioxolane), BOCHD-C3-DMA (4-(dimethylamino)-,9-(2-octylcyclopropyl)-1-[8-(2 octylcyclopropyl) octyl]nonyl ester), C12-200 (1,1′-[[2-[4-[2-[[2-[bis(2-hydroxydodecyl)amino]ethyl](2-hydroxydodecyl)amino]ethyl]-1-piperazinyl]ethyl]imino]bis-2-dodecanol), PNI 127 ((2R,3S,4S)-2-(((1,4-dimethylpiperidine-4-carbonyl)oxy)methyl)tetrahydrofuran-3,4-diyl (9Z,9′Z,12Z,12′Z)- bis(octadeca-9,12-dienoate)), PNI 516 ((Z)-3-(2-((1,17-bis(2-octylcyclopropyl)heptadecan-9-yl)oxy)-2-oxocthyl)-2-(pent-2-cn-1-yl)cyclopentyl 4-(dimethylamino)butanoate), PNI 550 (3-(2-((1,17-bis(2-octylcyclopropyl)heptadecan-9-yl)oxy)-2-oxocthyl)cyclopentyl 4- (dimethylamino)butanoate), PNI 560 ((Z)-3-(2-((1,17-bis(2-octylcyclopropyl)heptadecan-9-yl)oxy)-2-oxocthyl)-2-(pent-2-cn-1-yl)cyclopentyl 1,4- dimethylpiperidine-4-carboxylate), PNI 568 (1,17-bis(2-octylcyclopropyl)heptadecan-9-yl (Z)-2-(3-(2-(1-methylpyrrolidin-3-yl)acetoxy)-2-(pent-2-en-1- yl)cyclopentyl)acetate), PNI 580 ((2R,3S,4S)-2-(((4-(dimethylamino)butanoyl)oxy)methyl)tetrahydrofuran-3,4-diyl bis(2-hexyldecanoate)), PNI 659 ((2R,3R,4S)-3,4-bis((2-hexyldecyl)oxy)tetrahydrofuran-2-yl)methyl 4-(dimethylamino)butanoate), PNI 660 (((2R,3R,4S)-3,4-bis((2-hexyldecyl)oxy)tetrahydrofuran-2-yl)methyl 1,4-dimethylpiperidine-4-carboxylate), PNI 714 ((Z)-1-(2-(3-(2-(1-methylpyrrolidin-3-yl)acetoxy)-2-(pent-2-en-1-yl)cyclopentyl)acetoxy)-11-(2-octylcyclopropyl)undecan-3-yl 2-hexyldecanoate), PNI 721 ((2R,3S,4S)-2-((((2-(dimethylamino)ethyl)carbamoyl)oxy)methyl)tetrahydrofuran-3,4-diyl bis(2-hexyldecanoate)), PNI 722 (2-(((2R,3R,4S)-3,4-bis((2-hexyldecyl)oxy)tetrahydrofuran-2-yl) methoxy)-N,N-dimethylethan-1-amine), PNI 723 (((2R,3R,4S)-3,4-bis((2-hexyldecyl)oxy)tetrahydrofuran-2-yl)methyl 4-(diethylamino) butanoate), PNI 726 ((2R,3S,4S)-2-((3-(dimethylamino)propoxy)methyl)tetrahydrofuran-3,4-diyl bis(2-hexyldecanoate)), PNI 728 (((2R,3R,4S)-3,4-bis((2-hexyldecyl)oxy)tetrahydrofuran-2-yl)methyl (2-(dimethylamino)ethyl)carbamate), PNI 730 ((2R,3S,4S)-2-((2-(dimethylamino)ethoxy)methyl)tetrahydrofuran-3,4-diyl bis(2-hexyldecanoate)), PNI 761 ((2R,3S,4S)-2-((((3-(diethylamino)propyl)carbamoyl)oxy)methyl)tetrahydrofuran-3,4-diyl bis(2-hexyldecanoate)), PNI 762 (((2R,3R,4S)-3,4-bis((2-hexyldecyl)oxy)tetrahydrofuran-2-yl)methyl (2-(diethylamino)ethyl)carbamate), PNI 768 ((2R,3S,4S)-2-((((2-(dimethylamino)ethyl)carbamoyl)oxy)methyl)tetrahydrofuran-3,4-diyl bis(2- octyldodecanoate), PNI 769 (((2R,3R,4S)-3,4-bis((2-octyldodecyl)oxy)tetrahydrofuran-2-yl)methyl (2-(dimethylamino)ethyl)carbamate), PNI 771 (((2R,3S,4S)-2-((3-(dimethylamino)propoxy)methyl)tetrahydrofuran-3,4-diyl bis(2-octyldodecanoate)), PNI 825 (((2R,3R,4S)-3,4-bis((2-octyldodecyl)oxy)tetrahydrofuran-2-yl)methyl 1,4-dimethylpiperidine-4-carboxylate), or any combinations thereof. Additional non-limiting examples of suitable ionizable lipids are found in PCT Pub. Nos. WO20252589 and W021000041, which are incorporated herein for reference in their entireties.

The ionizable lipid may be present in the lipid nanoparticle composition or lipid nanoparticle in any suitable amount or concentration. In some preferred embodiments, ionizable lipid is present in the lipid compositions in a ratio of about 25 to about 50 mol percent, (“mol percent” means the percentage of a particular component in the lipid compositions, while the total mole of all the components in the lipid compositions is 100 mol %). The term “about” in this paragraph signifies a plus or minus range of 5 mol percent at increments of 0.1. For example, 28.7 mol percent would be in the claimed range of embodiments. In some embodiments, the ionizable lipid is present at a concentration of about 10 to about 75 mol %, e.g., about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, or about 70 mol %, about 75 mol %, or a concentration within a range defined by any two of the foregoing values.

In some embodiments, LNP composition includes a phospholipid (or referred to as “structural lipid” or “helper lipid” and used interchangeably throughout the disclosure). Any suitable structural lipid can be present in the lipid nanoparticle composition and lipid nanoparticle. A structural lipid supports the formation of particles during manufacture. In some embodiments, the structural lipid includes a phospholipid. In various embodiments, the structural lipid includes one or more neutrally charged, positively charged, or negatively charged molecules. In some embodiments, the structural lipid has a net negative charge. In some embodiments, the structural lipid has a net neutral charge. In some embodiments, the structural lipid has a net positive charge.

In some embodiments, the structural lipids include, but are not limited to, distearoylphosphatidylcholine (DSPC), dioleoylphosphatidylcholine (DOPC), dipalmitoylphosphatidylcholine (DPPC), bis(diphenylphosphino)ethane (DPPE), diacyl phosphatidylcholine, diacylphosphatidylethanolamine, diacylphosphatidylserine, diolcoyl-phosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-0-monom ethyl PE, 16-O-dimethyl PE, 18-1-trans PE, 1-stearoyl-2-oleoyl-phosphatidyethanolamine (SOPE), 1,2-dielaidoyl-sn-glycero-3- phophoethanolamine (trans DOPE), 1,2-dioleoyl-3-trimethylammonium propane (DOTAP), or combinations thereof. In one preferred embodiment, the phospholipid is distearoylphosphatidylcholine (DSPC). In preferred embodiments, the phospholipid is DOPE.

The structural lipid may be present in the lipid nanoparticle composition in any suitable amount. In some embodiments the structural lipid is present in the lipid nanoparticle composition at a concentration of about 1 to about 75 mol % or about 10 to about 55 mol %, e.g., about 1 mol %, about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60 mol %, about 65 mol %, or about 70 mol %, about 75 mol %, or a value within a range defined by any two of the aforementioned values. In some embodiments, there is no structural lipid.

“Stabilizer” or “stabilizing agent” is a term used to identify the agent that is added to the ionizable lipid, the phospholipid, and/or the sterol that form the lipid composition according to the invention. Non-limiting examples of stabilizing agents include, but are not limited to, Polyethyleneglycol (PEG), DMG-PEG2000 (1,2-dimyristoyl-rac-glycero-3-methoxypolyethylene glycol-2000), Polysorbates (Tweens), TPGS (Vitamin E polyethylene glycol succinate), Brij™ S20 (polyoxyethylene (20) stearyl ether), Brij™35 (Polyoxyethylene lauryl ether, Polyethyleneglycol lauryl ether), Brij™S10 (Polyethylene glycol octadecyl ether, Polyoxyethylene (10) stearyl ether), Myrj™52 (polyoxyethylene (40) stearate), or any combinations thereof. Additional non-limiting examples of stabilizing agents include those disclosed in PCT applications PCT/EP2024/075129, PCT/EP2024/075124, PCT/EP2024/075128, which are incorporated by reference herein in their entireties.

The stabilizing agent can be present in any suitable concentration. In some cases, the stabilizing agent has a concentration from about 0.1 mol % to about 10 mol %, e.g., about 0.1 mol %, about 0.2 mol %, about 0.3 mol %, about 0.4 mol %, about 0.5 mol %, about 0.6 mol %, about 0.7 mol %, about 0.8 mol %, about 0.9 mol %, about 1 mol %, about 1.2 mol %, about 1.4 mol %, about 1.6 mol %, about 1.8 mol %, about 2 mol %, about 2.5 mol %, about 3 mol %, about 3.5 mol %, about 4 mol %, about 4.5 mol %, about 5 mol %, about 5.5 mol %, about 6 mol %, about 6.5 mol %, about 7 mol %, about 7.5 mol %, about 8 mol %, about 8.5 mol %, about 9 mol %, about 9.5 mol %, about 10 mol %, or a concentration defined by a range of any two of the foregoing values. In some embodiments, there is no stabilizing agent.

Sterols are included in lipid compositions for certain applications, and lipid particles made therefrom include cholesterol, beta-sitosterol, 20-alpha-hydroxysterol, and/or phytosterol. The stabilizing agent can be present in any suitable concentration. In some cases, the stabilizing agent has a concentration from about 4 to 70 mol % of the lipid compositions, e.g., about 4 mol %, about 5 mol %, about 10 mol %, about 15 mol %, about 20 mol %, about 25 mol %, about 30 mol %, about 35 mol %, about 40 mol %, about 45 mol %, about 50 mol %, about 55 mol %, about 60mol %, about 65 mol %, about 70 mol %, or a concentration defined by a range of any two of the foregoing values. In some embodiments, sterol is present at about 20 to 40 mol % of the lipid compositions. In some embodiments, the sterol is cholesterol. In some embodiments, a modified sterol or synthetically derived sterol is present. In some embodiments, there is no sterol.

In some embodiments, the lipid nanoparticle composition comprises about 10 to about 75 mol % ionizable lipid, about 1 to about 75 mol % structural lipid, about 4 to about 70 mol % sterol, and 0 to about 3 mol % stabilizing agent. In some embodiments, the lipid nanoparticle composition comprises about 25 to about 50 mol % ionizable lipid, about 10 to about 55 mol % structural lipid, about 20 to about 40 mol % sterol, and 0 to about 3 mol % stabilizing agent.

In some embodiments, the lipid nanoparticle composition is used in the formation of a lipid nanoparticle in embodiments of the methods described herein. In some embodiments, the diameter of the lipid nanoparticle is about 15 nm to about 500 nm, e.g., about 15 nm, about 25 nm, about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, or about 500 nm, or a diameter defined by a range of any two of the foregoing values. Such diameters can be useful for improving the tissue targeting and biodistribution of the lipid nanoparticles. In some cases, the diameter of the lipid nanoparticle is more than about 10 nm, more than about 15 nm, more than about 20 nm, more than about 25 nm, more than about 30 nm, more than about 35 nm, more than about 40 nm, or more than about 45 nm. In some cases, the diameter of the lipid nanoparticle is less than about 700 nm, less than about 675 nm, less than about 650 nm, less than about 625 nm, less than about 600 nm, less than about 575 nm, less than about 550 nm, less than about 525 nm, less than about 500 nm, less than about 475 nm, less than about 450 nm, less than about 425 nm, less than about 400 nm, less than about 375 nm, less than about 350 nm, less than about 325 nm, or less than about 300 nm.

Embodiments of the lipid nanoparticle described herein can have any suitable polydispersity index. In some embodiments, the lipid nanoparticle has a polydispersity index of from about 0.01 to about 0.40, e.g., about 0.01, about 0.02, about 0.03, about 0.04, about 0.05, about 0.06, about 0.07, about 0.08, about 0.09, about 0.10, about 0.11, about 0.12, about 0.13, about 0.14, about 0.15 about 0.16, about 0.17, about 0.18, about 0.19, about 0.20, about 0.21, about 0.22, about 0.23, about 0.24, about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, or about 0.40, or polydispersity index defined by a range of any two of the foregoing values.

Embodiments of the lipid nanoparticle described herein can have any suitable encapsulation efficiency. Encapsulation efficiency refers to the percentage of nucleic acid that is successfully entrapped into the lipid nanoparticle. Encapsulation and complexation within LNPs can protect RNA from RNase digestion. The encapsulation efficiency does not have to be 100%. Presence of external RNA molecules (e.g., on the exterior surface of a liposome or LNP) or “naked” RNA molecules (RNA molecules not associated with a liposome or LNP) is acceptable. In some embodiments, the lipid nanoparticle has an encapsulation efficiency from about 50% to about 100%, e.g., about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 100%, or an encapsulation efficiency defined by a range of any two of the foregoing values.

Ultrafiltration and diafiltration (UF/DF) via tangential flow filtration (TFF) is an important unit operation in RNA manufacturing that may be employed. TFF steps may include UF/DF of a crude RNA mixture to remove impurities, as well as to condition the mRNA into a buffer suitable for the subsequent process step. A second UF/DF step may be performed to concentrate and condition the purified RNA into a storage buffer followed by bioburden filtration to make the bulk RNA drug substance. If the RNA is to be encapsulated into lipid nanoparticles (RNA-LNPs), a third UF/DF step may be needed to concentrate and buffer exchange into the cryopreservation buffer before sterile filtration and aseptic fill/finish of the product.

The lipid composition for preparing the LNP is a multi-component composition typically including an ionizable lipid, one or more of a structural lipid, sterol, or a stabilizer. A cationic ionizable lipid binds to an anionic nucleic acid under certain pH environment. Each individual component of the lipid composition may be customized based on desired outputs. For example, the component or concentration thereof may be modified based on a desired output. In some aspects, the present disclosure provides a method for producing a composition including a nanoparticle having at least one nucleic acid and at least one ionizable lipid. The method may include the steps of producing a nanoparticle having the at least one nucleic acid and the at least one ionizable lipid, filtering the nanoparticle from the reaction mixture by single-pass tangential flow filtration (SPTFF), and filtering retentate from the previous filtration step through a sterile filtration membrane to produce the composition. The nanoparticle may be produced by providing a first liquid composition having the at least one nucleic acid, providing a second liquid composition having the at least one ionizable lipid, and introducing the first liquid composition and the second liquid composition into at least one reactor, wherein the first liquid composition and/or the second liquid composition are introduced at predetermined flow rates, therefore generating the nanoparticle in a reaction mixture.

### EXAMPLE 1

### Method for Self-Amplifying mRNA Synthesis

The restriction digestion of a circular plasmid encoding SARS Covid spike protein was carried out according to manufacturer's instructions for BspQI (New England BioLabs Inc., catalog number R0712S) or Pmel (New England BioLabs Inc., catalog number R0560S), in vendor-prescribed buffers. The linearized vector was purified using phenol/chloroform/isoamyl alcohol-(25:24:1) and sodium acetate precipitation. Briefly, equal volumes of phenol/chloroform/isoamyl alcohol solution were added to the linearized vector, vortexed for 20 seconds and incubated at room temperature for 2 minutes. The mixture was spun at 12,000 g, after which the top aqueous phase containing the linearized vector was carefully pipetted into a clean RNase/DNase free tube and precipitated volumes of 100% ethanol were added, mixed well and spun at 12,000 g, after which the supernatant was removed carefully and the DNA pellet air dried and resuspended in nuclease free water. The concentration and purity of the linearized vector was determined using a NanoDrop™-spectrophotometer (VWR). In vitro transcription was carried out using HiScribe™ T7 High Yield RNA Synthesis Kit (New England BioLabs, Inc., catalog number E2040S) followed by linear DNA template digestion, performed using TURBO™ DNase (Thermofisher Scientific, catalog number AM2238), and the final in vitro transcribed self-amplifying RNA (saRNA) was capped using Vaccina Capping System™ (New England Biolabs Inc, catalog number M2080S). All these processes were performed according to the-manufacturer's protocols to generate the self amplifying RNA utilizing the DNA templates obtained from the vector linearization strategy. The purification of the capped saRNA was performed using standard NaOAc salt in ethanol precipitation and centrifugation, followed by 70% ethanol wash and resuspension of the RNA pellet in RNA storage solution (Thermofisher). This is described in PCT Pub. No. WO23057979 by Geall et al.

### EXAMPLE 2

### Microfluidic Mixing of Nucleic Acid and Lipid Compositions for Forming Lipid Nanoparticles (LNP)

Formulation of LNP was performed by rapidly mixing an organic solution (“organic phase”) including lipid components in an organic solvent (e.g. ethanol) with an aqueous buffer including a nucleic acid payload (“aqueous phase”) inside a microfluidic mixer designed to induce chaotic advection and/or provide a controlled mixing environment. In some cases, the microfluidic channels include herringbone features or are configured in a manner as shown in PCT Pub. Nos. WO2017117647, WO2016138175, WO2016176505, WO2018006166, or U.S. Pat. Nos. 10,835,878, 11,938,454, 10,597,291, 11,059,039, the contents of each are incorporated herein in their entireties. Particle sizes and “polydispersity index” (PDI) of the lipid particle were measured by dynamic light scattering (DLS). PDI indicates the width of the particle distribution. This is a parameter calculated from a cumulative analysis of the DLS measured intensity/autocorrelation function assuming a single particle size mode and a single exponential fit to the autocorrelation function. From a biophysical point of view, a PDI below 0.1 indicates that the sample is monodisperse. The particles produced by mechanical micromixers such as the NanoAssemblr™ Spark™ and NanoAssemblr™ Ignite™ (Cytiva, USA) are substantially homogeneous in size assuming all other variables are neutral. A lower PDI indicates a more homogenous population of lipid particles. Nucleic acid, payload, or nucleic acid therapeutic (NAT) as described above was diluted using sodium acetate buffer to the required concentration. LNP samples were then prepared as described by running both fluids using the NanoAssemblr™ Ignite instrument. Briefly, 63 μg of nucleic acids in sodium acetate buffer in a total volume of 0.75 mL was mixed with 0.25 mL of 12.5 mM lipid mix solution as required by the nitrogen to phosphate ratio (N/P) of 8, then LNP were diluted by in line dilution at 2:1 ratio in PBS.

### Nanoparticle (LNP) Characterization and Encapsulation

After the LNP were made as described above, the LNP particle size (hydrodynamic diameter of the particles) was determined by Dynamic Light Scattering (DLS) using a ZetaSizer™ Nano ZS™ (Malvern Instruments, UK) with a He/Ne laser of 633 nm as the light source. Data were measured from the scattered intensity data conducted in backscattering detection mode (measurement angle=173°). Measurements were an average of 10 runs of two cycles each per sample. Z Average size was reported as the particle size, and is defined as the harmonic intensity averaged particle diameter. Encapsulation efficiency was measured by a modified Ribogreen™ assay (Quanti-iT-RiboGreen™ RNA assay kit, Fisher). There was good encapsulation in all the formulations (see Table 2 for composition), with polydispersity (PDI) under 0.19. Post encapsulation steps were tangential flow filtration (TFF) concentration for about 40 minutes and diafiltration for the control TFF, and single pass TFF for the test cases.

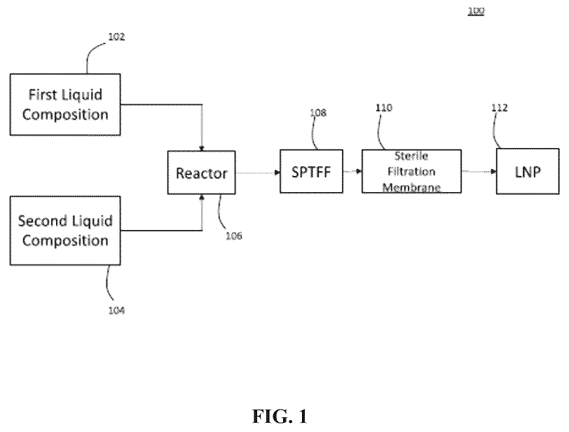

As illustrated in FIG. 1, a system in accordance with an embodiment of the invention is depicted. The system 100 may optionally include a first fluid source 102, e.g., vessel, container, bag, or the like, that contains a first composition having the at least one nucleic acid. The system 100 further optionally include a second fluid source 104 that contains a second composition having the at least one ionizable lipid. The system further includes a reactor 106. The first composition and the second composition may be introduced at a first flow rate and second flow rate, respectively, into reactor 106 or a mixer via one or more pumps (not shown). Bioreactors and/or mixers are used to carry out biochemical and biological processes and/or manipulate liquids and other products of such processes. The reactor 106 may be a rocking reactor, an adherent cell bioreactor, a stirred tank, an airlift bioreactor, a bubble column bioreactor, a fluidized bed bioreactor, a photobioreactor, or the like. The mixer may be a microfluidic mixer. In some embodiments, the first and second fluid sources are introduced into the microfluidic mixer, and lipid nanoparticles are collected from an outlet of the microfluidic mixer. In some embodiments, microfluidic mixing devices, which can involve mixing two or more types of fluids together uniformly in a microfluidic chip, such as the NanoAssemblr™ mixers including NanoAssemblr™ Spark™, NanoAssemblr™ Ignite™, NanoAssemblr™ Blaze™, NanoAssemblr™ GMP system, and NanoAssemblr™ commercial formulation system are used. In some embodiments, the lipid nanoparticles formed by using a microfluidic mixing device has an encapsulation efficiency from about 90 to about 100%.

In some embodiments, the first fluid source includes a payload in a first solvent. In some embodiments, the payload may include a nucleic acid. In some cases, the payload may include a therapeutic agent. The combination of the payload in the first solvent may be described as the aqueous phase. Any suitable first solvent may be used. Suitable first solvents include solvents in which the payload is soluble and that are miscible with the second solvent. In some embodiments, the first solvent comprises aqueous buffers. In some embodiments, the aqueous buffer includes a low pH buffer. In some embodiments, the low pH buffer includes a citrate or acetate buffer.

In some embodiments, the second stream includes embodiments of the lipid nanoparticle composition as described herein in a second solvent. The combination of the lipid nanoparticle composition and the second solvent may be described as the organic phase. Any suitable second solvent may be used. Suitable second solvents include solvents in which the ionizable lipids according to embodiments of the invention are soluble, and that are miscible with the first solvent. In some embodiments, the second solvent comprises one or more solvents, two or more solvents, three or more solvents, or four or more solvents. In some embodiments, the second solvent includes, but is not limited to, 1,4-dioxane, tetrahydrofuran, acetone, acetonitrile, dimethyl sulfoxide, dimethylformamide, acids, alcohols, or a combination thereof. In some embodiments, the second solvent comprises aqueous or anhydrous alcohols. In some cases, the alcohol includes a primary, secondary, or tertiary alcohol having from 1 to 12 branched or unbranched carbons (e.g., methanol, ethanol, n-propanol, isopropanol, n-butanol, 2-methyl 1-propanol, 2-butanol, 2-methylpropan-2-ol), or a combination thereof.

In one example, the first composition is introduced at a first flow rate and the second composition is introduced at a second flow rate. The first flow rate and the second flow rate may be the same. However, in other embodiments, the first flow rate may be greater or less than the second flow rate. The introduction of the first and second compositions in the reactor generates the LNP in a reaction mixture.

Any suitable flow ratio may be used to combine the first and second compositions. In some embodiments, the first composition and the second composition are combined using a flow ratio of about 1:1 (or 1) to about 20:1 (or 20) (aqueous phase: organic phase) by volume, e.g., about 1, about 2, about 3, about 4, about 5, about 6, or about 7, about 8, about 9, about 10, about 11, about 12, about 13, about 14, about 15, about 16, or about 17, about 18, about 19, about 20, or a flow ratio defined by a range of any two of the aforementioned values. In some cases, the flow ratio is more than about 0.5. In some cases, the flow ratio is less than about 30, less than about 28, less than about 26, less than about 24, less than about 22, less than about 20, or less than about 18. Any suitable N/P ratio may be used to combine the nanoparticle composition and the nucleic acid. The N/P ratio is the ratio of positively-charged polymer amine (N=nitrogen) groups to negatively-charged nucleic acid phosphate (P) groups. In some embodiments, the first liquid composition and the second liquid composition are combined at a N/P ratio from about 2 to about 20, e.g., about 2, about 3, about 4, about 5, about 6, about 7, about 8, about 9, about 10, about 11, about 12, about 13, about 14, about 15, about 16, about 17, about 18, about 19, or about 20, or at an N/P ratio defined by a range of any two of the aforementioned values. In some case, the N/P ratio is more than about 1, more than about 2, more than about 3, more than about 4, more than about 5, more than about 6, more than about 7, more than about 8, or more than about 9. In some case, the N/P ratio is less than about 40, less than about 38, less than about 36, less than about 34, less than about 32, less than about 30, less than about 28, less than about 26, less than about 24, less than about 22, less than about 20, less than about 18, less than about 16, less than about 14, less than about 12, or less than about 10.

Any suitable total flow rate can be used to combine the first liquid composition and the second liquid composition. In some embodiments, the first and second liquid compositions are combined with a total flow rate of the organic phase and aqueous phase from about 2 to about 2500 mL/min, e.g., about 2 mL/min, about 4 mL/min, about 6 mL/min, about 8 mL/min, about 10 mL/min, about 20 mL/min, about 40 mL/min, about 60 mL/min, about 80 mL/min, or about 100 mL/min, about 120 mL/min, about 140 mL/min, about 160 mL/min, about 180 mL/min, about 200 mL/min, about 220 mL/min, about 240 mL/min, about 260 mL/min, about 280 mL/min, about 300 mL/min, about 350 mL/min, about 400 mL/min, about 450 mL/min, or about 500 mL/min, about 550 mL/min, about 600 mL/min, about 650 mL/min, about 700 mL/min, about 750 mL/min, about 800 mL/min, about 850 mL/min, or about 900 mL/min, about 950 mL/min, about 1000 mL/min, about 1100 mL/min, about 1200 mL/min, about 1300 mL/min, about 1400 mL/min, about 1500 mL/min, about 1600 mL/min, about 1700 mL/min, about 1800 mL/min, about 1900 mL/min, about 2000 mL/min, about 2100 mL/min, about 2200 mL/min, about 2300 mL/min, about 2400 mL/min, about 2500 mL/min, or a total flow rate defined by a range of any two of the foregoing values. In some cases, the total flow rate is more than about 1 mL/min, 2 mL/min, 4 mL/min, 6 mL/min, 8 mL/min, 10 mL/min, 20 mL/min, or 40 mL/min. In some case, the total flow rate is less than about 3000 mL/min, less than about 2800 mL/min, less than about 2600 mL/min, less than about 2400 mL/min, less than about 2200 mL/min, less than about 2100 mL/min, less than about 2000 mL/min, less than about 1800 mL/min, less than about 1600 mL/min, less than about 1500 mL/min, less than about 1400 mL/min, less than about 1200 mL/min, less than about 1000 mL/min, or less than about 800 mL/min, In some embodiments, the first and second compositions are combined using a flow ratio from about 1:1 (or 1) to about 20:1 (or 20) by volume (aqucous phase: organic phase) at a N/P ratio from about 2 to about 20, and a total flow rate from about 2 to about 2500 mL/min. In some embodiments, the flow rate ratio is 3:1 (aqueous phase: organic phase) to optimize for a particular payload or molar ratio of lipid components.

Once generated, the LNP is introduced at a predetermined flux into the SPTFF filter 108. The flux may be measured in L/m²/hour (LMH). In embodiments, the flux is between 10 LMH and 200 LMH. In embodiments, the flux is between 12 LMH to 180 LMH, between 14 LMH to 160 LMH, between 16 LMH to 140 LMH, between 18 LMH to 120 LMH, between 20 LMH to 100 LMH, between 30 LMH to 90 LMH, between 40 LMH to 80 LMH, or between 50 LMH to 70 LMH. In embodiments, the fluxes are between 10 LMH and 20 LMH, between 20 LMH and 30 LMH, between 30 LMH and 40 LMH, between 40 LMH and 50 LMH, between 50 LMH and 60 LMH, between 60 LMH to 70 LMH, between 70 LMH and 80 LMH, between 80 LMH to 90 LMH, or between 90 LMH and 100 LMH. In preferred embodiments, the flow rates are selected from 10 LMH, 11 LMH, 12 LMH, 13 LMH, 14 LMH, 15 LMH, 16 LMH, 17 LMH, 18 LMH, 19 LMH, 20 LMH, 21 LMH, 22 LMH, 23 LMH, 24 LMH, 25 LMH, 26 LMH, 27 LMH, 28 LMH, 29 LMH, 30 LMH, 31 LMH, 32 LMH, 33 LMH, 34 LMH, 35 LMH, 36 LMH, 37 LMH, 38 LMH, 39 LMH, 40 LMH, 41 LMH, 42 LMH, 43 LMH, 44 LMH, 45 LMH, 46 LMH, 47 LMH, 48 LMH, 49 LMH, 50 LMH, 51 LMH, 52 LMH, 53 LMH, 54 LMH, 55 LMH, 56 LMH, 57 LMH, 58 LMH, 59 LMH, 60 LMH, 61 LMH, 62 LMH, 63 LMH, 64 LMH, 65 LMH, 66 LMH, 67 LMH, 68 LMH, 69 LMH, 70 LMH, 71 LMH, 72 LMH, 73 LMH, 74 LMH, 75 LMH, 76 LMH, 77 LMH, 78 LMH, 79 LMH, and 80 LMH. Integer and decimal values between and adjacent to the listed ranges are intended to be included in the embodiments.

As used herein, filtering may include flowing, passing, running, moving, or the like, through the SPTFF. The retentate from the SPTFF is then fed through a sterile filtration membrane 110 to remove particulate and contamination from the liquid composition. The sterile filtration membrane may include pores between about 0.01 μm and about 0.3 μm in size.

In embodiments, the pore size is about 0.01 μm to 0.02 μm, 0.02 μm to 0.03 μm, 0.03 μm to 0.04 μm, 0.04 μm to 0.05 μm, 0.05 μm to 0.06 μm, 0.06 μm to 0.07 μm, 0.07 μm to 0.08 μm, 0.08 μm to 0.09 μm, 0.09 μm to 0.1 μm, 0.1 μm to 0.2 μm, or 0.2 μm to 0.3 μm. This list provides non-limiting ranges of sizes within the given parameters. Decimal values between and adjacent to the listed ranges are intended to be included in the embodiments.

The produced composition is then directed to a receptacle 112 for containing the composition.

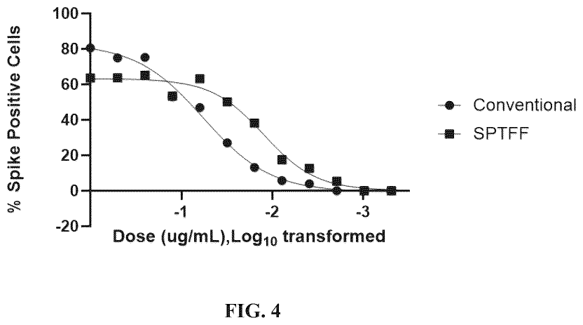

In one embodiment, a nanoparticle (e.g., lipid nanoparticle or “LNP”) may be processed using, for example, a Pall® Cadence™ SPTFF (Cytiva, USA). The lipid nanoparticle may include at least one nucleic acid and at least one ionizable lipid. The SPTFF method and system of producing LNP with maintained formulation potency which can reduce toxicity compared to traditional TFF. Said another way, by improving the potency of the LNP (as further illustrated in FIG. 4), less LNP is needed to achieve the same biologic effect, thus reducing toxicity from greater levels of LNP required by traditional TFF.

The SPTFF process of the instant disclosure produces LNP with higher in vitro potency than traditional TFF. In one example as shown in FIG. 4, the LNP shows a surprising multi-fold higher in vitro potency over LNP that were produced by TFF. Use of an SPTFF also reduces the downstream processing time, which prevents RNA degradation that may occur during a longer, standard TFF process. The downstream processing time may be reduced in SPTFF as the target compound may be pushed through the filters once, whereas conventional TFF may recirculate the target compound multiple times. Further, SPTFF may reduce hydrodynamic shear forces on the LNP, which may reduce degradation and preserve potency.

Additionally, the SPTFF configuration is modular and customizable based on desired outcomes. For example, the surface area of the cassettes used in the SPTFF may be adjusted. Increasing the surface area of the cassettes may lead to greater concentrations achieved. Further, the SPTFF configuration may be scaled up or scaled down, based on desired outcomes and operating parameters.

By producing LNP of higher potency than traditional TFF, SPTFF has the advantages of enabling a reduction in the dose of the LNP needed for therapeutic effect. A reduction in the dose may provide benefits against potential toxicity effects of larger doses of the LNP. The use of SPTFF allows the product to be moved through filters one time and may be pumped with a substantially smaller pump, compared to traditional TFF systems. Additionally, the SPTFF may use a lower working volume, which typically results in a higher product recovery, compared with conventional TFF.

In one embodiment, the Pall® Cadence™ SPTFF may be used to produce high in vitro potency of an LNP, or more than one LNP composition.

In a non-limiting example, a lipid composition V02, may be used. V02 may include 47.5 mol % ionizable lipid/12.5 mol % DOPE/38.5 mol % Cholesterol/1.5 mol % 1,2-dimyristoyl-rac-glycero-3-methoxypolyethylene glycol (DMG-PEG). The ionizable lipid in V02 can include any ionizable lipid as described in the instant disclosure. While specific compositions are provided, it is understood that these compositions may be modified without deviating from the inventive subject matter. DOPE may be replaced with DSPC or other helper lipids as described in the instant disclosure.

In another non-limiting example, a different lipid composition V46, may be used. V46 may include 28.7 mol % ionizable lipid (iL), 49.8 Mol % DSPC, 20 mol % cholesterol, and 1.5 mol % DMG-PEG. The ionizable lipid in V46 can include any ionizable lipid as described in the instant disclosure.

In another example, a yet another lipid composition, V47, may be used. V47 may include iL 29.1 mol %, DSPC 50.6 mol %, cholesterol 20.3 mol %, DMG-PEG 0 mol %. The ionizable lipid in V46 can include any ionizable lipid as described in the instant disclosure.

Additional non-limiting examples of lipid nanoparticle compositions for Example 1 are listed in Table 1.

TABLE 1
Non-limiting examples of lipid nanoparticle compositions as defined by
ionizable lipid/structural lipid/sterol/stabilizing agent mol % ratio, with the total mol %
of all the components in the lipid composition being 100 mol %.
Lipid Compositions (Chol stands for cholesterol)

10 mol % ionizable lipid (iL)/18.5 mol % DSPC/70 mol % Chol/1.5 mol % PEG-DMG
10.5 mol % iL/75.5 mol % DSPC/14 mol % Chol/0 mol% stabilizer
18 mol % iL/54 mol % DSPC/27 mol % Chol/1 mol % PEG-DMG
25 mol % iL/37 mol % DSPC/37 mol % Chol/1 mol % PEG-DMG
25 mol % iL/37.5 mol % DSPC/37.5 mol % Chol/0 mol % stabilizer
40 mol % iL/20 mol % DSPC/37.5 mol % Chol/2.5 mol % BRIJ ™ S10
40 mol % iL/15 mol % DSPC/44.3 mol % Chol/0.8 mol % TPGS
35 mol % iL/15 mol % DSPC/47.5 mol % Chol/2.5 mol % Brij 10
47.5 mol % iL/12.5 mol % DSPC/38.5 mol % Chol/1.5 mol % PEG-DMG
40 mol % iL/20 mol % DOPE/37.5 mol % Chol/2.5 mol % Tridecyl-D-maltoside
40 mol % iL/20 mol % DOPE/37.5 mol % Chol/2.5 mol % Polysorbate 20
40 mol % iL/20 mol % DOPE/37.5 mol % Chol/2.5 mol % Polysorbate 80
40 mol % iL/20 mol % DSPC/38.5 mol % Chol/1.5 mol % Polysorbate 80
40 mol % iL/20 mol % DSPC/39.5 mol % Chol/0.5 mol % TPGS
40 mol % iL/12.5 mol % DOPC/39.25 mol % Chol/0.75 mol % TPGS
40 mol % iL/12.5 mol % DPPC/46 mol % Chol/1.5 mol % TPGS
40 mol % iL/12.5 mol % DSPC/45 mol % Chol/2.5 mol % TPGS
40 mol % iL/20 mol % DSPC/37.7 mol % Chol/2.3 mol % TPGS
47.5 mol % iL/20 mol % DSPC/31.75 mol % Chol/0.75 mol % TPGS
47.5 mol % iL/20 mol % DSPC/31.5 mol % Chol/1 mol % TPGS
47.5 mol % iL/20 mol % DSPC/30 mol % Chol/2.5 mol % TPGS
40 mol % iL/20 mol % DSPC/39.5 mol % Chol/0.5 mol % polyoxyethylene (10) stearyl ether
40 mol % iL/20 mol % DSPC/37.5 mol % Chol/2.5 mol % % polyoxyethylene (10) stearyl ether
40 mol % iL/20 mol % DSPC/39.5 mol % Chol/0.5 mol % % polyoxyethylene (20) stearyl ether
40 mol % iL/40 mol % DOPE/17.5 mol % Chol/2.5 mol % Polyoxyethylene (40) stearate
40 mol % iL/20 mol % DOPE/37.5 mol % Chol/2.5 mol % Polyoxyethylene (40) stearate)
40 mol % iL/20 mol % DOPE/39 mol % Chol/1 mol % Polyoxyethylene (40) stearate)
40 mol % iL/20 mol % DOPE/20 mol % POPC/17.5 mol % Chol/2.5 mol %
Polyoxyethylene (40) stearate
40 mol % iL/12.5 mol % DSPC/46 mol % Chol/1.5 mol % PEG-DMG
40 mol % iL/12.5 mol % DOPC/46 mol % Chol/1.5 mol % PEG-DMG
40 mol % iL/12.5 mol % DSPC/46.5 mol % Chol/1% mol % DMG-PEG
40 mol % iL/12.5 mol % DPPC/46.5 mol % Chol/1% mol % DMG-PEG
40 mol % iL/13 mol % DOPE/46.5 mol % Chol/1% mol % DMG-PEG
40.0 mol % iL/15.0 mol % DSPC/42.5 mol % Chol/2.5 mol % TPGS
35.0 mol % iL/20.0 mol % DSPC/42.5 mol % Chol/2.5 mol % TPGS
36.2 mol % iL/18.0 mol % DSPC/44.0 mol % Chol/1.8 mol % TPGS
35.3 mol % iL/18.0 mol % DSPC/44.0 mol % Chol/2.7 mol % TPGS
40.0 mol % iL/15.0 mol % DSPC/42.3 mol % Chol/2.7 mol % TPGS
40.0 mol % iL/15.0 mol % DSPC/42.0 mol % Chol/3.0 mol % TPGS
39.5 mol % iL/15.0 mol % DSPC/42.0 mol % Chol/3.5 mol % TPGS
35.0 mol % iL/19.5 mol % DSPC/42.0 mol % Chol/3.5 mol % TPGS
47.5 mol % iL/12.5 mol % DOPE/38.5 mol % Chol/1.5 mol % PEG-DMG
47.5 mol % iL/12.5 mol % DSPC/38.5 mol % Chol/1.5 mol % PEG-DMG
47.5 mol % iL/12.5 mol % DPPC/38.5 mol % Chol/1.5 mol % PEG-DMG
47.5 mol % iL/12.5 mol % SOPC/38.5 mol % Chol/1.5 mol % PEG-DMG
47.5 mol % iL/12.5 mol % SOPC/38.5 mol % Chol/1.5 mol % TPGS
47.5 mol % iL/12.5 mol % DSPC/39 mol % Chol/1 mol % TPGS
47.5 mol % iL/12.5 mol % DSPC/39.25 mol % Chol/0.75 mol % TPGS
47.5 mol % iL/20 mol % DSPC/31.75 mol % Chol/0.75 mol % TPGS
47.5 mol % iL/20 mol % DSPC/31.5 mol % Chol/1 mol % TPGS
47.5 mol % iL/20 mol % DSPC/30 mol % Chol/2.5 mol % TPGS
50 mol % iL/10 mol % DSPC/38.5 mol % Chol/1.5 mol % PEG-DMG
50 mol % iL/10 mol % DOPC/38.5 mol % Chol/1.5 mol % PEG-DMG
50 mol % iL/10 mol % DOPE/38.5 mol % Chol/1.5 mol % PEG-DMG
50 mol % iL/10 mol % DSPE/38.5 mol % Chol/1.5 mol % PEG-DMG
50 mol % iL/10 mol % DSPC/37.5 mol % Chol/2.5 mol % TPGS
50 mol % iL/10 mol % DSPC/39 mol % Chol/1 mol % TPGS
50 mol % iL/12.5 mol % DSPC/36.5 mol % Chol/1 mol % TPGS
50 mol % iL/12.5 mol % DOPE/36.5 mol % Chol/1 mol % TPGS
50 mol % iL/12.5 mol % DOPC/36.5 mol % Chol/1 mol % TPGS
50 mol % iL/12.5 mol % DPPC/36.5 mol % Chol/1 mol % TPGS
50 mol % iL/12.5 mol % DSPC/36.75 mol % Chol/0.75 mol % TPGS
50 mol % iL/12.5 mol % DOPE/36.75 mol % Chol/0.75 mol % TPGS
50 mol % iL/12.5 mol % DOPC/36.75 mol % Chol/0.75 mol % TPGS
50 mol % iL/12.5 mol % DPPC/36.5 mol % Chol/0.75 mol % TPGS
50 mol % iL/20 mol % DSPC/29.25 mol % Chol/0.75 mol % TPGS
50 mol % iL/20 mol % DOPE/29.25 mol % Chol/0.75 mol % TPGS
50 mol % iL/20 mol % DOPC/29.25 mol % Chol/0.75 mol % TPGS
50 mol % iL/12.5 mol % DPPC/36.75 mol % Chol/0.75 mol % TPGS
50 mol % iL/20 mol % DSPC/28.5 mol % Chol/1.5 mol % TPGS
50 mol % iL/20 mol % DOPE/28.5 mol % Chol/1.5 mol % TPGS
50 mol % iL/20 mol % DOPC/28.5 mol % Chol/1.5 mol % TPGS
50 mol % iL/12.5 mol % DPPC/36 mol % Chol/1.5 mol % TPGS
50 mol % iL/20 mol % DSPC/27.5 mol % Chol/2.5 mol % TPGS
50 mol % iL/20 mol % DOPE/35 mol % Chol/2.5 mol % TPGS
50 mol % iL/20 mol % DOPC/35 mol % Chol/2.5 mol % TPGS
50 mol % iL/12.5 mol % DPPC/35 mol % Chol/2.5 mol % TPGS
50 mol % iL/10 mol % DSPC/38.5 mol % Chol/1.5 mol % Polysorbate 80
50 mol % iL/10 mol % DSPC/37.5 mol % Chol/2.5 mol % Polysorbate 80
50 mol % iL/10 mol % DSPC/39.5 mol % Chol/0.5 mol % Polysorbate 80
50 mol % iL/10 mol % DSPC/37.5 mol % Chol/2.5 mol % Polyoxyethylene (40) stearate
50 mol % iL/10 mol % DSPC/39.5 mol % Chol/0.5 mol % Polyoxyethylene (40) stearate
50 mol % iL/10 mol % DOPC/37.5 mol % Chol/2.5 mol % Polyoxyethylene (40) stearate)
50 mol % iL/10 mol % DOPE/37.5 mol % Chol/2.5 mol % Polyoxyethylene (40) stearate)
50 mol % iL/20 mol % DOPE/27.5 mol % Chol/2.5 mol % Polyoxyethylene (40) stearate
50 mol % iL/30 mol % DOPE/19 mol % Chol/1 mol % Polyoxyethylene (40) stearate)
50 mol % iL/10 mol % DSPC/37.5 mol % Chol/2.5 mol % polyoxyethylene (20) stearyl ether
50 mol % iL/10 mol % DSPC/37.5 mol % Chol/2.5 mol % polyoxyethylene (10) stearyl ether
50 mol % iL/10 mol % DSPC/38.5 mol % Chol/1.5 mol % polyoxyethylene (10) stearyl ether
50 mol % iL/10 mol % DSPC/37.5 mol % Chol/2.5 mol % Polyoxyethylene (4) lauryl ether
50 mol % iL/10 mol % DSPC/37.5 mol % Chol/2.5 mol % Polyoxyethylene (23) lauryl ether
54 mol % iL/10 mol % DOPC/35 mol % Chol/1.0 mol % PEG-DMG
61.1 mol % IL/0.5 mol % DSPC/38.4 mol % Chol/0 mol % stabilizer
75 mol % IL/19.1 mol % DSPC/4.4 mol % cholesteryl hemisuccinate/1.5 mol % PEG-DMG

In embodiments, the ionizable lipid includes PNI 127, PNI 516, PNI 550, PNI 560, PNI 568, PNI 580, PNI 659, PNI 660, PNI 714, PNI 721, PNI 722, PNI 723, PNI 726, PNI 728, PNI 730, PNI 761, PNI 762, PNI 768, PNI 769, PNI 771, PNI 825, or combinations thereof.

The nucleic acid therapeutic (NAT) or payload, are described above.

The LNP including nucleic acid of the present disclosure may include the ionizable lipid in an amount of 10 mol % to 60 mol %, with the total mol % of the components in the lipid composition being 100 mol %. The ionizable lipid may be included in an amount of 10 mol % to 30 mol % with respect to the total 100 mol % of all components in the lipid composition. When including the ionizable lipid in an amount of 10 mol % or greater as above, LNP having a greater potency may be manufactured, however ionizable lipid when bound to nucleic acid may contribute to toxicity more than other LNP components. SPTFF may allow usage of a lower % of ionizable lipids or a lower dose by maintaining potency of the LNP when compared to conventional TFF that requires significant recirculation.

The ionizable lipid and the nucleic acid may have a weight ratio in a range of 3:1 to 75:1, e.g., about 3:1, about 4:1, about 5:1, about 6:1, about 7:1, about 8:1, about 9:1, about 10:1, about 15:1, about 20:1, about 25:1, about 30:1, about 35:1, about 40:1, about 45:1, about 50:1, about 55:1, about 60:1, about 65:1, about 70:1, about 75:1, or at a weight ratio defined by a range of any two of the aforementioned values. In some embodiments, the weight ratio is in a range 5:1 to 50:1.

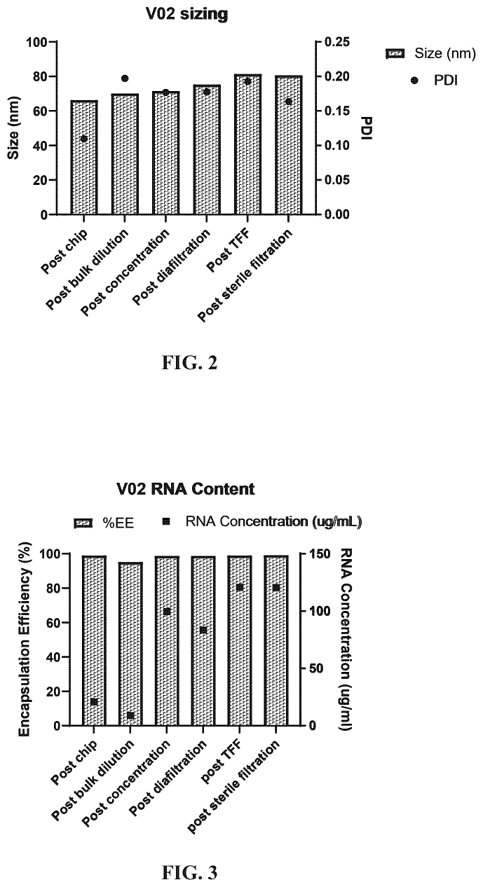

As illustrated in the data shown in FIGS. 2, 3 and 5-8, the method for producing the liquid composition comprising the nanoparticle may start with mixing a lipid composition and a payload in a reactor to provide a “post chip” sample. In a non-limiting example, the method may start with mixing a 50 mg batch of V02, TFR 200 saRNA in 100 mM sodium acetate buffer pH3, with a 3:1 aqueous: organic phase flow rate ratio. The saRNA may be between 0.01 and 0.1 mg/mL, with a volume between 25 mL and 100 mL, preferably 0.084 mg/mL at 37.5 mL. A non-limiting example of the lipid mix composition may include ionizable lipid PNI 516((Z)-3-(2-((1,17-bis(2-octylcyclopropyl)heptadecan-9-yl)oxy)-2-oxoethyl)-2-(pent-2-en-1-yl)cyclopentyl 4-(dimethylamino) butanoate). The lipid composition including PNI 516 may be at 12.5 mM. It is understood that the quantity, molarity, and acidity of the reagents and the like are described herein as non-limiting examples and may be modified. For example, in some embodiments, other ionizable lipids described throughout the disclosure can be used. Additionally, the lipid composition may include one or more of helper lipid, sterol, or stabilizer.

“Post Chip” is intended to mean “after initial LNP formation”. A chip may be a mixing region, a reaction vessel, a T-mixer, a herringbone, a toroidal microfluidic mixing cartridge, a bifurcating microfluidic mixing cartridge, and the like. “After initial LNP formation” therefore means “after the convergence of the first and second liquid compositions, where initial LNP formation occurs”. Encapsulation of the nucleic acid within LNP occurs during the initial LNP formation. After SPTFF, the after initial formation processed and retained by the SPTFF is referred to as a retentate.

The method may then move to a dilution step of diluting the “post chip” sample to provide a “post bulk dilution” sample. The dilution may include an in-line dilution of the LNP in the reactor after the LNP is formed. In some embodiments, the dilution is between 1.5 and 5 times dilution of the post chip sample. Examples of solvent that can be used for in-line dilution include, but are not limited to, phosphate buffered saline (PBS). In embodiments, the dilution provides a diluted LNP such that the volume percent of the organic solvent (e.g., ethanol) in the total volume of the post bulk dilution sample may be less than 10% ethanol. In embodiments, the dilution may include an additional manual dilution of LNP after the initial in-line dilution step. The manual dilution may be between 1.5 and 5 times dilution so that the ethanol percent in the diluted LNP is further lowered to less than 8%, e.g., less than 1%, 2%, 3%, 4%, 5%, 6%, or 7% ethanol. In embodiments, the post bulk dilution sample includes less than 3% ethanol.

The method then involves loading the post bulk dilution sample into the SPTFF system. The method may include a first concentration step, in which the post bulk dilution sample is concentrated as the post bulk dilution sample flows through the SPTFF system to a predetermined volumetric concentration factor (VCF) and to provide a “post concentration” sample including a concentrated LNP. When the method is performed at higher pressure, the VCF may decrease. When the method is performed at lower pressure, the VCF may increase. In some embodiments, the post bulk dilution sample only flows through the SPTFF system once (i.e., without recirculation) in the first concentration step.

The method may further include subjecting the post concentration sample to a diafiltration and/or buffer exchange to provide the LNP into a final formulation buffer and to provide a “post diafiltration” sample. In some embodiments, the final formulation buffer is a cryobuffer. In some embodiments, the final formulation buffer is different from a buffer used in the composition including the at least one nucleic acid. In some embodiments, the post concentration sample only flows through the SPTFF system once (i.e., without recirculation) in the diafiltration step. In embodiments, the SPTFF system includes an in-line diafiltration module such as Cadence™ inline diafiltration module (Cytiva, Marlborough, USA).

The method may further include subjecting the post diafiltration sample to a second concentration step in which the post diafiltration sample in the final formulation buffer or cryobuffer is concentrated as the post diafiltration sample flows through the SPTFF system to provide a retentate or a “post TFF” sample. In some embodiments, the post diafiltration sample only flows through the SPTFF system once (i.e., without recirculation) in the second concentration step.

The method may further include subjecting the post TFF sample to sterile filtration to provide a “post sterile filtration” sample. In embodiments, the sterile filtration includes filtering the retentate produced from the SPTFF system through a sterile filtration membrane.

In an embodiment, a sterile filtration apparatus includes a membrane or membranes, a column, depth filtration, cartridge filtration, or a syringe filtration. Membrane filtration uses a porous membrane to remove microorganisms and particles from a fluid. The membrane acts as a physical barrier, allowing the passage of solvent or desired substances while blocking contaminants. Depth filters are composed of multiple layers of different materials, such as cellulose fibers or activated carbon. They work by trapping particles through a combination of mechanisms like adsorption, absorption, and/or size exclusion. Cartridge filtration uses cartridge filters that includes a cylindrical housing filled with a filter medium such as activated carbon, pleated polypropylene, or ceramic. They are commonly used for small-scale sterile filtration in laboratories or pharmaceutical manufacturing. Syringe filtration uses syringe filters that are small, disposable filters designed to attach to a syringe for rapid small-volume filtration. They are frequently used in laboratory settings for clarifying or sterilizing liquids.

In some embodiments, the SPTFF system includes a concentration module configured to perform the first and second concentration of the LNP sample. The SPTFF system may further include a diafiltration module configured to perform the diafiltration and/or buffer exchange of the LNP sample. In embodiments, all of the first concentration, diafiltration, the second concentration occur in the same SPTFF system. With SPTFF system, the nanoparticle is run through the equipment one time, compared to conventional TFF, where the nanoparticle is recirculated through the equipment multiple times.

FIG. 2 illustrates a graph of the size and polydispersity index of LNP including lipid composition V02 with DSPC as helper lipid (“V02 LNP”) prepared by a number of post processing methods to illustrate the type of characteristics that are important for LNP. The size is measured in nanometers. The size of the V02 LNP may be between 20 nm and 100 nm. A polydispersity index may be used to indicate the homogeneity of an LNP, for example, the particle size distribution of the LNP. A small polydispersity index generally indicates a narrow particle size distribution. The V02 LNP may have a polydispersity index from about 0.1 to about 0.3. The size and the polydispersity index may be measured at various points of the method, for example, post chip, post bulk dilution, post concentration, post diafiltration, post SPTFF, and post sterile filtration.

Table 2 is the data summary for various processes with respect to V02 LNP. The size, PDI and encapsulation levels are on par with traditional TFF methods as shown in Table 3.

TABLE 2
LNP Physical Characteristics

| | | | Encapsulation |
|---|---|---|---|
| Sample Description | Size (nm) | PDI(%) | (%) |
| diluted Blaze (Starting), | 86 | 0.246 | 99 |
| “post bulk dilution” | | | |
| SPTFF concentrated, | 71 | 0.211 | 99 |
| “post concentration” | | | |
| SPTFF diafiltration, | 75 | 0.212 | 99 |
| “post diafiltration” | | | |
| SPTFF collected, “Post | 80 | 0.198 | 999 |
| TFF” | | | |

TABLE 3
Multipass TFF was also optimized to establish a reliable
baseline or control for the SPTFF method.

| Sample | | | Encapsulation |
|---|---|---|---|
| Description | Size (nm) | PDI(%) | (%) |
| 3x dilution (“Post | 70 | 0.207 | 99 |
| chip”) | | | |
| 8x dilution (“post | 65 | 0.172 | 99 |
| bulk dilution”) | | | |
| TFF concentrated | 73 | 0.256 | 99 |
| 6.0x-6.5x (“post | | | |
| concentration”) | | | |
| TFF diaflitration | | | |
| (“Post | | | |
| diafiltration”) | | | |
| TFF collected | 92 | 0.343 | 98 |
| (“Post TFF”) | | | |
| After sterile | 77 | 0.171 | 98 |
| filtration (“Post | | | |
| sterile filtration”) | | | |

Between the two processes, encapsulation, size, and polydispersity were generally consistent.

FIG. 3 is a graph of the encapsulation efficiency and RNA concentration of V02 LNP. The encapsulation efficiency may be measured as a percentage and may be an amount of the target compound or payload that is encapsulated within an LNP, relative to the initial total amount of target compound/payload used in the preparation of an LNP. The target compound may be a nucleic acid. In this non-limiting example, the payload is a RNA. The encapsulation efficiency and RNA concentration may be measured at various points of the method, for example, post chip, post bulk dilution, post concentration, post diafiltration, post SPTFF, and post sterile filtration. As illustrated in FIG. 3, the encapsulation efficiency and RNA concentration increase greatly after the filtration steps.

FIG. 4 is a graph showing the comparison of percentage of spike positive cells per dose transformed of conventional tangential flow filtration and single-pass tangential flow filtration according to embodiments of the invention.

As illustrated, significantly more potent LNP are produced by using the SPTFF process of the instant disclosure compared with the conventional TFF. Advantageously, the SPTFF process of the instant disclosure may be able to use therapeutically “weaker” payloads or those compositions more difficult to effectively filter as a result of the maintained potency and reduced shear, respectively. This allows for a wider variety of payloads to be used, creating a wider array of drugs.

Further, additional advantages for the SPTFF method according to the invention is the simplified manufacturing process requiring less adjustments to the way one would operate the equipment. SPTFF can also greatly reduce the time needed for the process. Conventional TFF requires the composition be recirculated multiple times through the filtration system, whereas the composition only goes through the filtration system once in the SPTFF system. Further, the pump needed in the SPTFF system may be substantially smaller than the pump needed in the conventional TFF system.

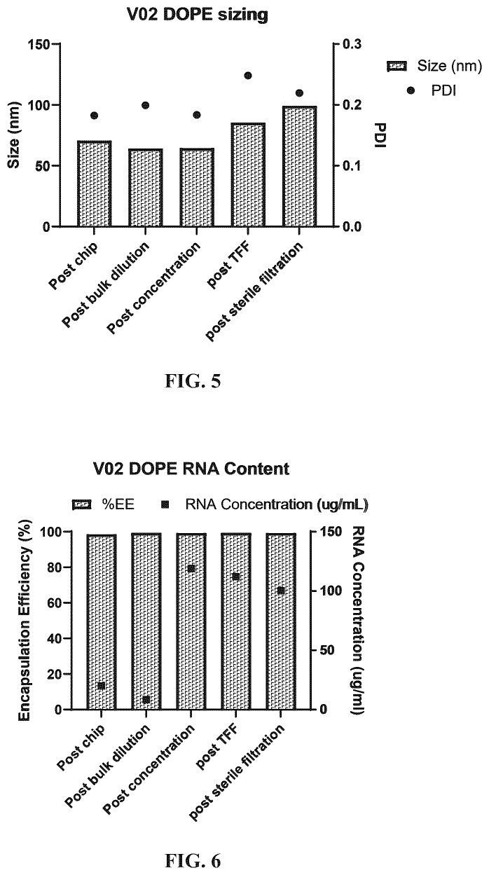

FIG. 5 is a graph illustrating the size and polydispersity index of V02 LNP with DOPE as a helper lipid component (“V02 DOPE LNP”). The size of the V02 DOPE LNP particles may be between 50 nm and 150 nm. The V02 DOPE LNP may have a polydispersity index from about 0.1 to about 0.3. The size and the polydispersity index may be measured at various points of the method, for example, post initial LNP formation, post bulk dilution, post concentration, post diafiltration, post SPTFF, and post sterile filtration.

FIG. 6 is a graph illustrating encapsulation efficiency and RNA concentration of V02 DOPE LNP. The encapsulation efficiency and RNA concentration may be measured at various points of the method, for example, post initial LNP formation, post bulk dilution, post concentration, post diafiltration, post SPTFF, and post sterile filtration. The encapsulation efficiency and RNA concentration increase greatly after the filtration steps.

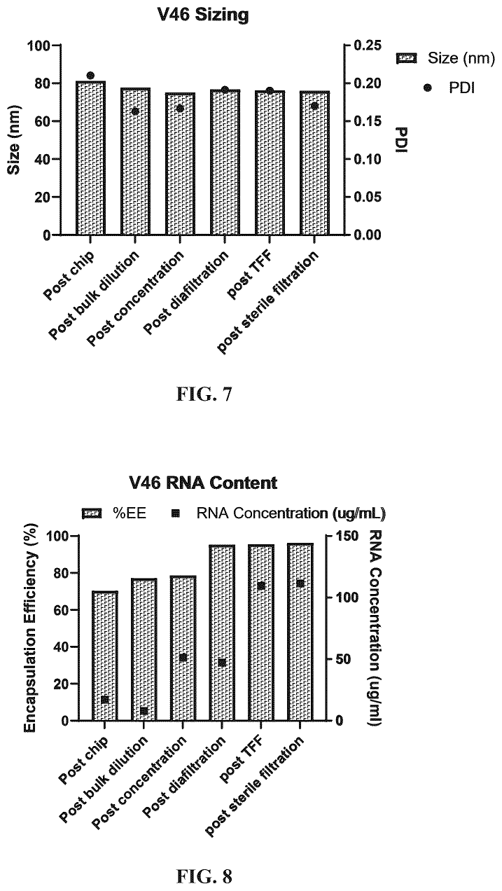

FIG. 7 is a graph illustrating the size and polydispersity index of V46 LNP with DSPC as a helper lipid (“V46 LNP”). The size of the V46 LNP particles may be between 50 nm and 100 nm. The V46 LNP may have a polydispersity index from about 0.1 to about 0.3. The size and the polydispersity index may be measured at various points of the method, for example, post initial LNP formation, (as defined above, post mixing) post bulk dilution, post concentration, post diafiltration, post SPTFF, and post sterile filtration.

FIG. 8 is a graph illustrating encapsulation efficiency and RNA concentration of V46 LNP. The encapsulation efficiency and RNA concentration may be measured at various points of the method, for example, post initial LNP formation, post bulk dilution, post concentration, post diafiltration, post SPTFF, and post sterile filtration. The encapsulation efficiency and RNA concentration increase greatly after the filtration steps.

Table 4 includes data illustrating the potency of V02 and V46 LNP prepared by various inputs compared to traditional TFF. Scouting runs were first used to identify the optimal range of volumetric concentration factors (VCF), feed fluxes (L/m²/hour), and feed pressures (PSI). Operation runs at the optimized range demonstrated the higher potency of LNP manufactured with the SPTFF process versus the conventional multiple pass TFF. V02 and V46 are different lipid compositions o as described herein and showcased a range of competence for the new process.

The various inputs may include volumetric concentration factor, the feed flux, and the feed pressures. The feed flux may be associated with the speed at which the compound is fed through the SPTFF. The feed flux may be measured in L/m²/hour. The feed pressure is measured in pounds per square inch (psi). The feed pressure may be between 10 and 60 psi, preferably between 17 and 35 psi. As illustrated, a baseline or scouting reading for V02 and V46 LNP is determined. For the baseline V02 LNP, a volumetric concentration factor between 10-16 is used with a feed flux of 56-141 LMH and feed pressures of 20-60 psi are used. For one variation of V02 LNP, the volumetric concentration factor of approximately 10 is used, with a feed flux of 21.3 LMH, and a feed pressure of 17 psi. With these inputs, the LNP potency was approximately 4.2× higher than traditional TFF. The EC50 for these parameters was 6 ng/ml. EC50 is the concentration that gives half-maximal response, and a lower value represents a more potent LNP. A second variation of V02 was used with a volumetric concentration factor of 14, a feed flux of 80 LMH, and a feed pressure of 35 psi. With these inputs, the LNP potency was approximately 4.6× higher than traditional TFF. The EC50 for these parameters was 12.4 ng/mL.

TABLE 4

| | VCF | | | Potency | |
|---|---|---|---|---|---|
| | (volumetric | | Feed | comparison | |
| | concentration | Feed Flux | pressures | to TFF | EC50 |
| Formulation | factor) | (Liter/m²/Hour) | (psi) | control | (ng/mL) |
| V02 SPTFF | 1-16 | 56-141 | 20-60 | N/A | N/A |
| scouting | | | | | |
| V02 SPTFF | 10.1 | 21.3 | 17 | 4.2x | 6 |
| operation | | | | | |
| V02 conventional | 16 | 450 | 25 | N/A | 25 |
| TFF operation | | | | | |
| V02 SPTFF | 13.1 | 80 | 35 | 4.6x | 12.4 |
| operation #2 | | | | | |
| V02 conventional | 16 | 450 | 25 | N/A | 57.1 |
| TFF operation #2 | | | | | |
| V46 SPTFF | 7-18 | 22-100 | 10-60 | N/A | N/A |
| scouting | | | | | |
| V46 SPTFF | 9.3 | 40 | 22 | N/A | 2.3 |
| operation | | | | | |

For the scouting of V46 LNP, a volumetric concentration factor between 7-18 is used with a feed flux of between 22-100 LMH and feed pressures of between 10-60 psi. For one variation of V46 LNP, the volumetric concentration factor of 9.3 is used, with a feed flux of 40 LMH, and a feed pressure of 22 psi. With these inputs, the EC50 for these parameters was 2.3 ng/mL.

In one embodiment, a method is provided for producing a composition that includes a nanoparticle with at least one nucleic acid and at least one ionizable lipid, the method may include the steps of: introducing a first composition that includes the at least one nucleic acid and a second composition that includes the at least one ionizable lipid into at least one reactor, filtering the nanoparticle from the reaction mixture via a single-pass tangential flow filter, and filtering retentate through a sterile filtration membrane to produce the composition.

The filtering step may include one or more of flowing, passing, running, or moving the nanoparticles from the reaction mixture to the single-pass tangential flow filter. The at least one nucleic acid may include ribonucleic acid (RNA). The nucleic acid may include be one or more of circular, linear or closed deoxyribonucleic Acid (DNA), linear or circular ribonucleic acid (RNA). In embodiments, the nucleic acid includes a circular RNA, messenger RNA (mRNA), transfer RNA (tRNA), ribosomal RNA (rRNA), small nuclear RNA (snRNA), microRNA (miRNA), small interfering RNA (siRNA), a self-amplifying RNA (saRNA), guide or targeting RNA, or combinations thereof. Proteins or enhancers associated with an application of the nucleic acid may also be included.

The predetermined flow rate may include a first flow rate for the first liquid composition and a second flow rate for the second liquid composition, wherein the first flow rate may be different from the second flow rate. The ratio between the two flow rates (first: second flow rate or second: first flow rate) may be 1:2, 1:3, 1:1, 2:1, 3:1, 4:1, for example.

In one embodiment, a system for producing a composition is provided. The system includes a reactor and a single-pass tangential filtration system. In some embodiments, the system further includes a first fluid source that includes a first composition having at least one nucleic acid and a second fluid source that includes a second composition having at least one ionizable lipid. The reactor is configured to introduce the first composition at a first flow rate to the second composition at a second flow rate to generate a nanoparticle in a reaction mixture. The single-pass tangential filtration system is configured to filter the nanoparticle from the reaction mixture at a defined feed flux. In embodiments, the system may further include a sterile filtration membrane configured to filter retentate produced from the single-pass tangential flow filtration system.

In one embodiment, the feed flux is between 10 L/m²/hour and 200 L/m²/hour. Flux can be defined as the rate of volume flow across a unit area.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the inventive subject matter without departing from its scope. While the embodiments described herein are intended to define the parameters of the inventive subject matter, they are by no means limiting and are example embodiments. Many other embodiments will be apparent to one of ordinary skill in the art upon reviewing the above description. The scope of the inventive subject matter should, therefore, be determined with reference to the appended clauses, along with the full scope of equivalents to which such clauses are entitled. In the appended clauses, the terms “including” and “in which” are used as the plain-English equivalents of the respective terms “comprising” and “wherein.” Moreover, in the following clauses, the terms “first,” “second,” and “third,” etc. are used merely as labels, and are not intended to impose numerical requirements on their objects. Further, the limitations of the following clauses are not written in means-plus-function format and are not intended to be interpreted based on 35 U.S.C. § 112(f), unless and until such clause limitations expressly use the phrase “means for” followed by a statement of function void of further structure.

This written description uses examples to disclose several embodiments of the inventive subject matter, including the best mode, and also to enable one of ordinary skill in the art to practice the embodiments of inventive subject matter, including making and using any devices or systems and performing any incorporated methods. The patentable scope of the inventive subject matter is defined by the clauses, and may include other examples that occur to one of ordinary skill in the art. Such other examples are intended to be within the scope of the clauses if they have structural elements that do not differ from the literal language of the clauses, or if they include equivalent structural elements with insubstantial differences from the literal languages of the clauses.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “comprises,” “including,” “includes,” “having,” or “has” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

1. A method for producing a composition that includes a nanoparticle with at least one nucleic acid and at least one ionizable lipid, the method comprising:
introducing a first composition that includes the at least one nucleic acid and a second composition that includes the at least one ionizable lipid into at least one reactor, wherein the first composition and the second composition are introduced at a first flow rate and a second flow rate, respectively, therefore generating the nanoparticle in a reaction mixture; and
filtering the nanoparticle from the reaction mixture via a single-pass tangential flow filtration system at a feed flux to provide a retentate, to produce the composition.

2. The method of clause 1, further including filtering the retentate through a sterile filtration apparatus before or after filtering the nanoparticle from the reaction mixture via the single-pass tangential flow filtration system.

3. The method of claim 1, wherein filtering the nanoparticle further includes:
concentrating the reaction mixture to provide a concentrated sample;
subjecting the concentrated sample to an in-line diafiltration to provide a post diafiltration sample; and
concentrating the post diafiltration sample to provide the retentate.

4. The method of claim 3, wherein concentrating the reaction mixture comprises flowing the reaction mixture via the single-pass tangential flow filtration system once and without recirculation.

5. The method of claim 1, wherein the nanoparticles in the reaction mixture are filtered in a batch-wise fashion or continuously.

6. The method of claim 1, wherein the at least one nucleic acid includes a linear or a circular ribonucleic acid (RNA) or deoxyribonucleic acid (DNA).

7. The method of claim 1, wherein the at least one nucleic acid includes a circular RNA, messenger RNA (mRNA), transfer RNA (tRNA), ribosomal RNA (rRNA), small nuclear RNA (snRNA), microRNA (miRNA), small interfering RNA (siRNA), a self-amplifying RNA (saRNA), guide or targeting RNA, or combinations thereof and/or wherein the at least one nucleic acid further includes a protein or enhancer associated with an application of the nucleic acid.

8. The method of claim 1, wherein the second composition further includes one or more of a phospholipid, sterol, or a stabilizing agent.

9. The method of claim 8, wherein the second composition comprises about 10-75 Mol % ionizable lipid, about 1-75 Mol % phospholipid, about 4-70 Mol % sterol, and about 0-3 Mol % stabilizer, wherein the total mol % of components in the second composition is 100 mol %.

10. A system for producing a composition comprising:
a reactor configured to combine a first composition at a first flow rate from a first fluid source and a second composition at a second flow rate from a second fluid source to generate a nanoparticle in a reaction mixture, wherein the first composition comprises at least one nucleic acid and the second composition comprises at least one ionizable lipid; and
a single-pass tangential flow filtration system configured to filter the nanoparticle from the reaction mixture at a feed flux to provide a retentate.

11. The system of claim 10, further optionally including a sterile filtration apparatus in fluidic communication with the single-pass tangential flow filtration system and configured to filter the retentate provided from the single-pass tangential flow filtration system.

12. The system of claim 10, wherein the single-pass tangential flow filtration system is configured to flow the reaction mixture through once and without recirculation.

13. The system of claim 10, wherein the single-pass tangential flow filtration system is configured to:
concentrate the reaction mixture to provide a concentrated sample;
flow the concentrated sample via an in-line diafiltration to provide a post diafiltration sample; and
concentrate the post diafiltration sample to provide the retentate.

14. The system of claim 10, wherein the single-pass tangential flow filtration system includes a concentration module and a diafiltration module.

15. The system of claim 10, wherein the single-pass tangential flow filtration system is configured to filter the nanoparticles from the reaction mixture in a batch-wise fashion or continuously.

16. The system of claim 10, wherein the at least one nucleic acid includes a linear or a circular ribonucleic acid (RNA) or deoxyribonucleic Acid (DNA).

17. The system of claim 10, wherein the at least one nucleic acid includes a circular RNA, messenger RNA (mRNA), transfer RNA (tRNA), ribosomal RNA (rRNA), small nuclear RNA (snRNA), microRNA (miRNA), small interfering RNA (siRNA), a self-amplifying RNA (saRNA), guide or targeting RNA, or combinations thereof and/or wherein the at least one nucleic acid further includes a protein or enhancer associated with the at least one nucleic acid.

18. The system of claim 10, wherein the second composition further includes one or more of a phospholipid, sterol, or a stabilizing agent.

19. The system of claim 18, wherein the second composition comprises about 10-75 Mol % ionizable lipid, about 1-75 Mol % phospholipid, about 4-70 Mol % sterol, and about 0-3 Mol % stabilizer, wherein the total mol % of components in the second composition is 100 mol %.

20. The method of claim 1, wherein the method is free of an evaporation step.
